(19) United States
(12) Patent Application Publication
(10) Pub. No.: US 2002/0002200 A1
(43) Pub. Date: Jan. 3, 2002

(54) Novel diphenylethylene compounds

(76) Inventors: Bishwagit Nag, Fremont, CA (US); Debendranath Dey, Fremont, CA (US); Satyanarayana Medicherla, Cupertino, CA (US); Partha Neogi, Fremont, CA (US)

Correspondence Address: REGINALD J. SUYAT, Fish & Richardson P.C., Suite 100, 2200 Sand Hill Road, Menlo Park, CA, 94025, US

(21) Appl. No.: 09/777,551

(22) Filed: Feb. 5, 2001

Related U.S. Application Data
(63) Continuation-in-part of application No. 09/642,618, filed on Aug. 17, 2000.
(60) Provisional application No. 60/180,340, filed on Feb. 4, 2000.

(51) Int. Cl. A61K 31/36; A61K 31/277; A61K 31/192; A61K 31/165; A61K 31/12; C07C 69/76; C07C 49/747; C07D 317/44

(52) U.S. Cl. 514/464; 514/521; 514/532; 514/617; 514/679; 549/443; 558/388; 560/37; 560/57; 564/171; 568/325

(57) ABSTRACT

Novel dephenylethylene compounds that are administered orally to decrease circulating concentrations of glucose are provided. The effect on insulin resistant rats is also shown. The effects on lipid and leptin concentrations are also shown. The compounds are orally effective anti-diabetic agents that may normalize glucose and lipid metabolism in subjects with diabetes.

### FIELD OF THE INVENTION

The field of the invention is novel diphenylethylene compounds and their use for the treatment of diabetes and related conditions.

### BACKGROUND OF THE INVENTION

Extracts of the leaves, flowers, and gum of the tree Pterocarpus marsupium Roxb. (Leguminosae), also known as the Indian Kino Tree, have been used traditionally to treat diarrhea, toothaches, fever, and urinary and skin infections. Extracts of the bark have been long regarded as useful for treating diabetes. Manickam et al. (J. Nat. Prod. 1997; 60:609-610) reported some hypoglycemic activity of a naturally occurring pterostilbene, trans-1-(3,5-dimethoxyphenyl)-2-(4-hydroxyphenyl)-ethylene, isolated from the heartwood of Pterocarpus marsupium. However, this pterostilbene is insoluble in water and has not been shown to be efficacious in the treatment of diabetes.

The causes of Type I and Type II diabetes are still unknown, although both genetic and environmental factors seem to be involved. Type I diabetes (or insulin-dependent diabetes) is an autoimmune disease in which the responsible autoantigen is still unknown. Subjects with Type I diabetes need to take insulin parenterally to survive. Type II diabetes (also referred to as non-insulin dependent diabetes mellitus, NIDDM) is a metabolic disorder resulting from the body's inability either to produce enough insulin or to properly use the insulin that is produced. Insulin secretion and insulin resistance are considered the major metabolic defects, but the precise genetic factors involved remain unknown.

Subjects with diabetes usually have one or more of the following defects:

Under-production of insulin by the pancreas

Over-secretion of glucose by the liver

Defects in glucose transporters

Desensitization of insulin receptors

Defects in metabolic breakdown of polysaccharides

In addition to insulin, which is administered parenterally, currently available medications used for diabetes include the 4 classes of oral hypoglycemic agents listed in the following table.

| | Marketed | Mechanism of | |
|---|---|---|---|
| Class | Drugs | Action | Limitations |
| Sulfonylureas | First | Signals beta | Development |
| | generation: 2 | cells to release | of resistance |
| | Second | more insulin | Hypoglycemia |
| | generation: 3 | | |
| Biguanides | Metformin | Reduces hepatic | Improves |
| | | glucose | sensitivity |
| | | production | to insulin |
| | | | Adverse |
| | | | hepatic effects |
| | | | Lactic acidosis |
| | | | Unwanted |
| | | | gastrointestinal |
| | | | effects |
| Glucosidase | Acarbose | Reduces glucose | Works only |
| inhibitors | | absorption | after meals |
| | | from gut | GI side effects |
| Thiazolidinediones | Troglitazone | Reduce insulin | Not effective |
| | (withdrawn) | resistance | in 25% of |
| | Rosiglitazone | | subjects |
| | Pioglitazone | | Require |
| | | | frequent liver |
| | | | function tests |
| | | | Have very long |
| | | | onset of action |
| | | | Cause weight |
| | | | gain |

As is apparent from the above table, there are disadvantages to the currently available antidiabetic agents. Accordingly, there is continuing interest in identifying and developing new agents—particularly orally administered, water-soluble compounds—that can be used to treat diabetes.

In addition to the pterostilbene discussed above, (−)-epicatechin has also been isolated from Pterocarpus marsupium by Sheehan et al. (J. Nat. Prod. 1983; 46:232) and reported as having a hypoglycemic effect (see also Chakravarthy et al. Life Sciences 1981; 29:2043-2047). Other phenolic compounds have been isolated from Pterocarpus marsupium by Maurya et al. (J Nat. Prod. 1984; 47:179-181), Jahromi et al. (J. Nat. Prod. 1993; 56:989-994), and Maurya et al. (Heterocycles 1982; 19:2103-2107).

### SUMMARY OF THE INVENTION

A class of compounds having the general formulas (I) and (II) have glucose-lowering activity.

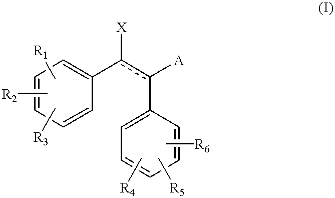

In compounds of Formula I, the bond represented by the dotted line may be an optional double bond, and the geometry across the bond may be either E or Z.

In formulas I and II A=—COOR, —CONR′R″, —CN, or —COR₇ wherein R,R′,R″ and R₇ are defined as below;

X=H, OH, or C₁-C₁₀ linear or branched alkyl or alkenyl groups that may be substituted with COOR, carbonyl, or halo;

R=H, linear or branched C₁-C₂₀ alkyl or aryl or aralkyl, Na, K, or other pharmaceutically acceptable counter-ion such as calcium, magnesium, ammonium, tromethamine, and the like;

R₁, R₂, R₃, R₄, R₅, R₆ and R₇ are independently H; C₁-C₂₀ linear or branched alkyl or alkenyl groups optionally substituted, COOR; NR′R″ or CONR′R″, where R′ and R″ may be independently H or C₁-C₂₀ linear or branched alkyl or aryl; OH; C₁-C₂₀ alkoxy; C₁-C₂₀ acylamino; C₁-C₂₀ acyloxy; C₁-C₂₀ alkanoyl; C₁-C₂₀ alkoxycarbonyl; halo; NO₂; SO₂R′″; CZ₃ wherein each Z is independently a halo atom, H, alkyl, chloro or fluoro-substituted alkyl; or SR′″, where R′″ may be H or linear or branched C₁-C₂₀ alkyl; or R₂ and R₃ together, or R₅ and R₆ together, may be joined to form methylenedioxy or ethylenedioxy groups.

In compounds of Formula II, the bond represented by the dotted line may be an optional double bond, and the geometry across the bond may be either E or Z; and the naphthyl group may be linked at an α or β position.

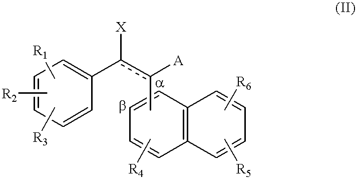

Pharmaceutical compositions of compounds of the formula I and/or II are provided for treatment of diabetes comprising a therapeutically effective amount of the compound in a pharmaceutically acceptable carrier.

A method of treating diabetes is also provided comprising a step of orally administering to a subject suffering from a diabetic condition a therapeutically effective amount of a compound of formula I and/or II.

### BRIEF DESCRIPTION OF THE DRAWINGS

FIG. 1 is a graph showing that compound Ia lowers blood glucose concentrations in rats with streptozotocin-induced diabetes.

FIG. 2 is a graph showing that compound Ia lowers blood glucose concentrations in ob/ob mice.

FIGS. 3A, B, C, are graphs showing that compound Ia lowers insulin, triglyceride, and free fatty acid concentrations in ob/ob mice.

FIG. 4 is a graph showing that compound Ia lowers blood glucose concentrations in db/db mice.

FIGS. 5A, B, C, are graphs showing that compound Ia lowers triglyceride and free fatty acid concentrations in db/db mice.

FIG. 6 is a graph showing that compound Ia orally administered is more effective than IP administered in maintaining lowered blood glucose concentrations.

FIGS. 7A, B are graphs showing that compound Ia lowers blood glucose concentrations in female obese (fa/fa) Zucker rats without affecting body weight.

FIGS. 8A, B, C, D are graphs showing that compound Ia improves the glucose tolerance of female obese fa/fa Zucker rats.

FIGS. 9A, B are graphs showing that compound Ia lowers serum insulin, and increases leptin concentrations, in female obese Zucker fa/fa rats.

FIG. 10 is a graph showing that compound Ia lowers cholesterol, triglyceride, and free fatty acid concentrations in female Zucker fa/fa rats.

FIGS. 11A, B, C, D are graphs showing that compound Ia (20 mg/kg daily) lowers the insulin, triglyceride, free fatty acid, and cholesterol concentrations in male obese Zucker fa/fa rats.

FIGS. 12A, B are graphs showing that compound Ia does not lower glucose concentrations in normal animals.

FIGS. 13A, B are graphs showing that compound Ia stimulates glucose uptake in adipocytes.

FIGS. 14A, B, C, D are graphs showing that compound Ia increases GLUT-1 and GLUT-4 transporters in 3T3-L1 cells.

FIGS. 15A, B ,C show, respectively, results of a lethal effect study on Swiss Webster mice by administration of compound Ia of dosages of 16.7, 167, and 333 mg/kg/BW on day zero.

FIG. 16 is a graph showing that Wortmannin (a known PI-3 kinase inhibitor) blocks compound Ia mediated glucose uptake in adipocytes.

FIG. 17 is a graph showing compound Ia stimulates the phosphorylation of the insulin receptor β subunit and insulin receptor substrate 1 in CHO.IR cells.

FIG. 18 is a graph showing compound Ia does not stimulate the phosphorylation of the IGF-1 receptor in CHO.IGF-1 R cells.

FIG. 19 is a graph showing that compound Ia stimulates the phosphorylation of Akt (protein kinase B) in CHO.IR cells.

FIG. 20 is an illustration of a Western blot showing that Wortmannin inhibits compound Ia stimulated Akt phosphorylation.

FIG. 21 is a graph showing that compound Ia does not up-regulate the expression of PPAR-γ in 3T3-L1 adipocytes.

FIG. 22 summarizes the results of binding studies that show that compound Ia is not an agonist of nuclear PPARs.

FIG. 23 is a graph showing compound Ia inhibits the binding of insulin to the insulin receptor.

FIGS. 24A, 24B are graphs showing that two isomers Ia and Ib (E and Z) stimulate rapid glucose uptake in rat adipocytes.

FIGS. 25A, B are graphs showing the results of pharmacokinetic studies of compound Ia in Sprague-Dawley rats.

FIG. 26 is a chart summmarizing the results of the toxicology studies conducted with compound Ia under Good Laboratory Practice regulations.

### DESCRIPTION OF THE PREFERRED EMBODIMENTS

Compounds of Formulas I and II are provided by synthetic methods generally known in the art. See Pettit et al., J. Nat. Prod., 1988, 51(3), pp 517-527 for a method for making E-isomers similar to Ia and Kessar et al., Indian J. of Chem., 1981, 20B, pp1-3 for making Z-isomers similar to Ib.

Preferred are compounds of formula I in which A=—COOR; R₁, R₄, R₆=H, and R₂ and R₃=methoxy (OCH₃) and R=H and R₅=OH and the dashed line represents a carbon-carbon double bond in either the E or Z configuration. More preferred are compounds of formula I in which R₁, R₄, R₆=H, and R₂=OCH₃ in the 3-position, and R₃=OCH₃ in the 5-position, and R₅=OH in the 4-position and the dashed line represents a carbon-carbon double bond in either the E or Z configuration, X=H, and R=H or a pharmaceutically acceptable cation such as lithium, sodium, potassium, calcium, magnesium, ammonium, tromethamine and the like, which may be introduced orally or parenterally to a subject.

Also preferred are compounds of formula II in which A=—COOR; R₁, R₄, R₆=H, and R₂ and R₃=methoxy (OCH₃) and R=H and R₅=OH and the dashed line represents a carbon-carbon double bond in either the E or Z configuration. More preferred are compounds of formula II in which R₁, R₄, R₆=H, and R₂=OCH₃ in the 3-position and the dashed line represents a carbon-carbon double bond in either the E or Z configuration; X=H, and R=H or a pharmaceutically acceptable cation such as lithium, sodium, potassium calcium, magnesium ammonium, tromethane and the like, which may be introduced orally or parenterally to a subject.

In general, compounds of formula I may be prepared by the condensation of: A) Appropriately substituted (R₁, R₂, R₃) benzaldehyde or phenylketone with appropriately substituted (R₄, R₅, R₆) phenylacetic acid or phenylacetic acid ester; B) Appropriately substituted (R₁, R₂, R₃) benzaldehyde or phenylketone with appropriately substituted (R₄, R₅, R₆) phenylacetamide; C) Appropriately substituted (R₁, R₂, R₃) benzaldehyde or phenylketone with appropriately substituted (R₄, R₅, R₆) phenylacetonitrile.

In general, compounds of formula II may be prepared by the condensation of: A) Appropriately substituted (R₁, R₂, R₃) benzaldehyde or phenylketone with appropriately substituted (R₄, R₅, R₆) naphthylacetic acid or naphthylacetic acid ester; B) Appropriately substituted (R₁, R₂, R₃) benzaldehyde or phenylketone with appropriately substituted (R₄, R₅, R₆) naphthylacetamide; C) Appropriately substituted (R₁, R₂, R₃) benzaldehyde or phenylketone with appropriately substituted (R₄, R₅, R₆) naphthylacetonitrile.

In Scheme I, the synthesis of compound Ia is shown as an exemplary synthesis. An exemplary synthesis of conversion of Ia to its Z-isomer is shown in Scheme II.

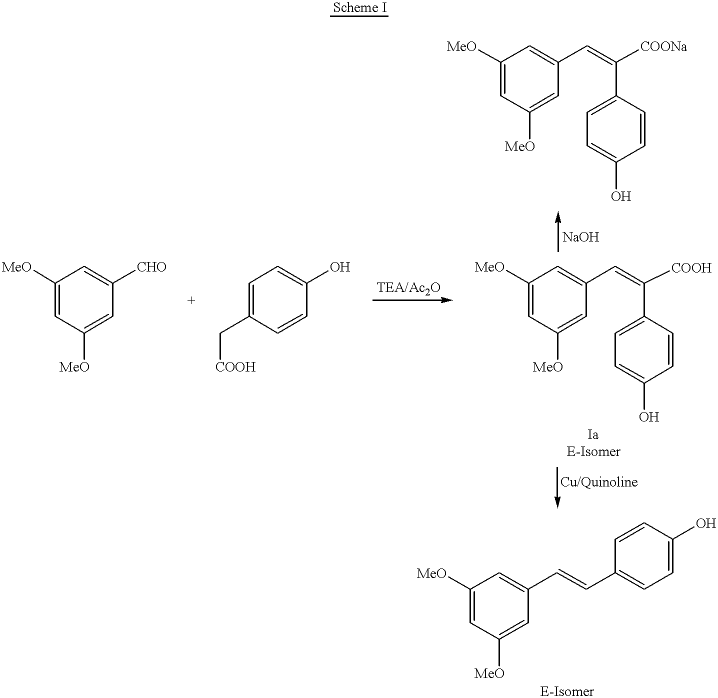

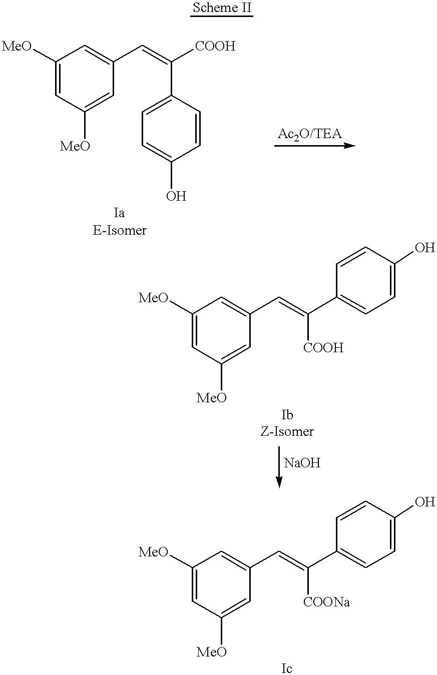

In the compounds of the formulas I, and II, the alkyl groups may be linear or branched including but not limited to methyl, ethyl, propyl, isopropyl, sec-butyl, n-butyl, pentyl, isopentyl, and the like. Alkenyl groups of 1 to 20 carbon atoms include but are not limited to, ethylene, propylene, butylene, isobutylene, and the like. Aryl groups include phenyl, and other multi-ring aromatic structures. Alkoxy includes methoxy, ethoxy propoxy, isopropoxy, n-butoxy, isobutoxy, methylenedioxy, ethylenedioxy and the like. Halo includes bromo chloro, fluoro, iodo.

Acylamino includes the group

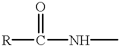

wherein R is hydrogen, alkyl, or aryl.

Acyloxy includes the group

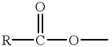

wherein R is hydrogen, alkyl or aryl.

Alkanoyl includes the group

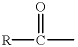

wherein R can be hydrogen, alkyl or aryl.

Alkoxycarbonyl includes the group

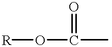

wherein R can be alkyl, aryl, or aralkyl.

The compounds according to the present invention may be combined with pharmaceutically acceptable carriers and vehicles in various compositions suitable for oral or parenteral delivery. The particularly preferred form of composition is either an orally administered capsule or solution in which the compound is delivered in water, saline, or a phosphate buffer; or lyophilized powder in the form of tablets or capsules also containing various fillers and binders. The effective dosages of the compound in a composition will be selected by those of ordinary skill in the art and may be determined empirically.

The compounds of the present invention are useful for the treatment of diseases characterized by the presence of elevated blood glucose concentrations, i.e., hyperglycemic disorders such as diabetes mellitus, including both Type I and Type II diabetes, as well as other disorders related to hyperglycemia, such as obesity, increased cholesterol concentrations, and renal disorders.

“Treatment” means that the compound is administered at least to reduce the blood glucose concentration in the subject suffering from the hyperglycemic disorder; the compound may also reduce insulin or lipid concentrations or both. The compound is administered in an amount sufficient to reduce blood glucose concentration to an acceptable range, wherein an acceptable range means within about +10% of the normal average blood glucose concentration for a subject of that species. A variety of subjects in addition to humans may be treated with the compounds to reduce blood glucose concentrations, such as livestock, valuable or rare animals, and pets. The compounds may be administered to the subject suffering from the hyperglycemic disorder using any administration technique, including intravenous, intradermal, intramuscular, subcutaneous, or oral. However the oral route of administration is particularly preferred. The dosage delivered to the subject will depend on the route by which the compound is delivered, but generally ranges from 5 to 500 mg for a 70-kg human, typically about 50 to 200 mg for a 70-kg human.

Of particular interest are methods of treating human hyperglycemic disorders such as diabetes (both Type I and Type II) in which the compound is administered to the human suffering from the hyperglycemic disorder to at least reduce the blood glucose concentration of the subject to about the normal blood glucose range for a human; the compound may also reduce insulin or lipid concentrations or both.

The following examples are offered by way of illustration, and are not intended to limit the invention in any way.

### EXAMPLE 1

### Synthesis of E-3-(3,5-dimethoxy-phenyl)-2-(4-hydroxy-phenyl)-acrylic Acid

To a mixture of 3,5-dimethoxybenzaldehyde (30 mmol) and p-hydroxyphenyl acetic acid (30 mmol) was added 5 mL acetic anhydride and 2.5 mL of triethylamine (TEA). After being stirred at 130-140° C. for 24 h, the mixture was cooled to room temperature and quenched with 25 mL concentrated HCl and extracted with CH₂Cl_{2.} The organic extract was further extracted with 1 N NaOH, then the NaOH extract was washed with CH₂Cl₂, and the aqueous layer was acidified with concentrated HCl and washed with water to obtain the crude product. Crude product was recrystallized from ethanol/water to yield the acid Ia.

Four lots of Ia (E-isomer) prepared as described above were separated in 40 μl samples by HPLC on an Intersil ODS-3 (GL Sciences) column, 250×4.6 mm, and eluted with 62% v eluent A and 38% v eluent B. Eluent A is 0.1% formic acid in water; B is 0.1% formic acid in ACN. All samples showed a major amount of the E-isomer, with a minor amount of Ib (Z-isomer) at relative retention time 1.073±0.001. By this method, presence of the Z-isomer was estimated to be from 0.27% to 3.09% in these samples.

### Synthesis of Z-3-(3,5-dimethoxy-phenyl)-2-(4-hydroxy-phenyl)-acrylic Acid

The Z-acid Ib was synthesized by a procedure described by Kessar et al., supra, who showed that E-a-phenyl cinnamic acids can be converted to similar Z-a-phenyl cinnamic acids by prolonged heating under basic conditions. The E-acid Ia (1.2 g, 4.0 mmol) was dissolved in a mixture of triethylamine (5.0 ml) and acetic anhydride (0.5 ml) and heated to reflux for 24 hours. The mixture was then cooled, diluted with ethyl acetate, and extracted sequentially first with 5% HCl (aqueous) then with 2 N NaOH and water. The combined basic aqueous solutions were acidified to a pH of 5 with acetic acid and cooled, and the solid was filtered. The filtrate was further acidified with concentrated HCl. Precipitation occurred upon cooling. The solid was collected by filtration and washed with fresh water. The solid compound was air dried to yield Ib.

Both isomers were subjected to NMR, pKa, HPLC, and UV spectral analysis.

E-Isomer.

The free acid form of the E-isomer showed a chemical shift for the olefinic proton (in DMSO-d₆) of δ7.59. The free acid has a melting point of 225-227° C. and a pKa of 6.2.

Z-Isomer.

The ¹H NMR analysis of the Z-isomer produced as described above showed the chemical shift of the olefinic proton to be 66.81 as a free acid in DMSO-d₆. The free acid form has a melting point of 135-137° C. and a pKa of 5.3.

Comparison of Isomers Produced.

The chemical shifts of the olefinic protons of the E- and Z-isomers prepared as described above are δ7.59 and δ6.81, respectively. As reported by Gadre and Marathe, Synth Commun 1988; 18:1015-1027, the compound with the higher chemical shift of the olefinic proton is the E-isomer, and the respective shifts seen with the prepared compounds are in agreement with that.

The analysis of the Perkin reaction product of phenyl acetic and benzaldehyde (a similar compound), indicates that the pKa of the isomers of a-phenyl cinnamic acid are 6.1 for the E-isomer and 4.8 for the Z-isomer, Fieser L F and Williamson K L, Exp. In Org. Chem (3rd ed.), Lexington, Mass.; Heath and Company, 1955, p182. Accordingly, between the two isomers, the one having the higher pKa is the E-isomer.

HPLC and UV Spectral Analysis

The reverse-phase HPLC analysis of E- and Z-isomers was performed by a linear gradient using a 0.1% formic acid/water/acetonitrile system on a G.L. Sciences Intersil ODS-3 column (250×4.6 mm, 5 μm), monitored at 280 nm. In this system, the E- and Z-isomers were eluted at 17.4 and 17.9 min, respectively.

Each isomer has a distinct UV spectrum. The Amax values for the E-isomer are 227 nm and 284 nm, and those for the Z-isomer are 221 nm and 303 nm.

Synthesis of E-4-[2-(3,5-dimethoxy-phenyl)-vinyl]-phenol

To decarboxylate Ia, 3 g of Cu powder and 30 mL of quinoline were added to 1 g of Ia under N₂ and refluxed with stirring for 4 h (still under N₂). The reaction mixture was filtered, acidified with concentrated HCl, and extracted with CH₂Cl_{2.} The organic layer was washed with aqueous saturated NaCl, dried and concentrated. The decarboxylated product was purified by flash chromatography over silica gel.

Synthesis of E-3-(3,5-dimethoxy-phenyl)-2-(4-hydroxy-phenyl)-acrylic Acid Sodium Salt

To convert the acid Ia to the sodium salt, NaOH solution was added to 1 g of Ia under room temperature; the mixture was shaken and freeze-dried to give the sodium salt of Ia.

### EXAMPLE 2

### Synthesis of 3-(3,4-Dimethoxy-phenyl)-2-(4-hydroxy-phenyl)-acrylic Acid

To a mixture of 3,4-dimethoxybenzaldehyde (9.97 g, 60 mmol) and p-hydroxyphenyl acetic acid (10.0 g, 65 mmol) under argon atmosphere was added acetic anhydride (12 mL) and triethylamine (8.0 mL, 58 mmol). The mixture was stirred at 140° C. for 18 h. The reaction mixture was cooled to 5° C. and dichloromethane (100 mL) was added. To this yellow suspension concentrated HCl (20 ml) was added and the suspension stirred for 20 min. The solid separated was filtered, dissolved in aqueous sodium hydroxide (2M, 225 mL) and re-precipitated with concentrated HCl (40 mL). Yellow solid was filtered and washed with water (2×30 mL) and the wet solid was recrystallized from a water-ethanol mixture.

¹H NMR (DMSO-d₆): δ12.38 (br. 1H), 9.47 (br, 1H), 7.61 (s, 1H), 6.96 (d, J=8.4 Hz, 2H), 6.81-6.77 (overlapped, 4H), 6.54 (br, 1H), 3.40 (s, 3H) and 3.37 (s, 3H).

### Synthesis of 3-(3,4-Dimethoxy-phenyl)-2-(4-fluoro-p-phenyl)-acrylic Acid

To a mixture of 3,5-dimethoxybenzaldehyde (4.98 g, 30 mmol) and p-fluorophenyl acetic acid (4.62 g, 30 mmol) under argon atmosphere was added acetic anhydride (5 mL) and triethylamine (5.0 mL, 36 mmol). The mixture was stirred at 140° C. for 18 h. The reaction mixture was cooled to room temperature and diethyl ether (100 mL) was added. The ether solution was further cooled to 10° C. and acidified with concentrated HCl (35 mL). The aqueous layer was discarded and the organic layer was extracted with aqueous sodium hydroxide solution (2M, 3×75 mL). Aqueous layers were pooled together and acidified with concentrated HCl (40 mL). The resulting precipitate was filtered, washed with water (2×30 mL) and recrystallized from a water-ethanol mixture.

¹H NMR (DMSO-d₆): δ 12.73 (br, 1H), 7.69 (s, 1H), 7.22 (d, J=7.2 Hz, 4H), 6.38 (t, J=2.5 Hz, 1H), 6.23 (d, 2.5 Hz, 2H), and 3.33 (s, 6H).

### Synthesis of 2-(4-Acetylamino-phenyl)-3-(3,5-dimethoxy-phenyl)-acrylic Acid

To a mixture of 3,5-dimethoxybenzaldehyde (2.5 g, 15 mmol) and p-aminophenyl acetic acid (2.28 g, 15 mmol) under argon atmosphere was added acetic anhydride (5 mL) and triethylamine (3.4 mL, 24 mmol). The mixture was stirred at 140° C. for 2 h. The reaction mixture was cooled to room temperature and chloroform (50 mL) was added. The chloroform solution was further cooled to 10° C. and acidified with concentrated HCl (10 mL). The aqueous layer was discarded and the organic layer was extracted with aqueous sodium hydroxide solution (2M, 3×50 mL). Aqueous layers were pooled and acidified with concentrated HCl to pH 1. The resulting precipitate was filtered, washed with water (2×30 mL) and recrystallized from a water-ethanol mixture.

¹H NMR (DMSO-d₆): δ 12.70 (br, 1H), 10.04 (s, 1H), 7.63 (s, 1H), 7.54 (d, J=7.5 Hz, 2H), 7.08 (d, J=7.5 Hz, 2H), 6.36(t, J=2.4 Hz, 1H), 6.25(d, J=2.4 Hz, 2H), 3.56(s, 6H) and 2.04(s, 3H).

### Synthesis of 3-(3,4-Dimethoxy-phenyl)-2-(4-hydroxy-phenyl)-propionic Acid

3-(3,4-Dimethoxy-phenyl)-2-(4-hydroxy-phenyl)-acrylic acid was dissolved in ethanol (100 mL) and palladium-charcoal (10%, 50% wet, 0.3 g) was added. The mixture was stirred overnight under hydrogen at room temperature. The reaction mixture was filtered through a bed of Celite® diatomaceous earth and the solvent evaporated.

¹H NMR (DMSO-d₆): δ 12.15 (br, 1H), 9.22 (br, 1H), 7.10 (d, J=8.6 Hz, 2H), 6.67 (d, J=8.6 Hz, 2H), 6.31 (d, J=2.2 Hz, 2H), 6.27 (t, J=2.2 Hz, 1H), 3.70 (s, 6H), 3.14 (dd, J=13.3 and 8.6 Hz, 2H), and 2.80 (dd, J=13.3 and 8.6 Hz, 2H).

### EXAMPLE 3

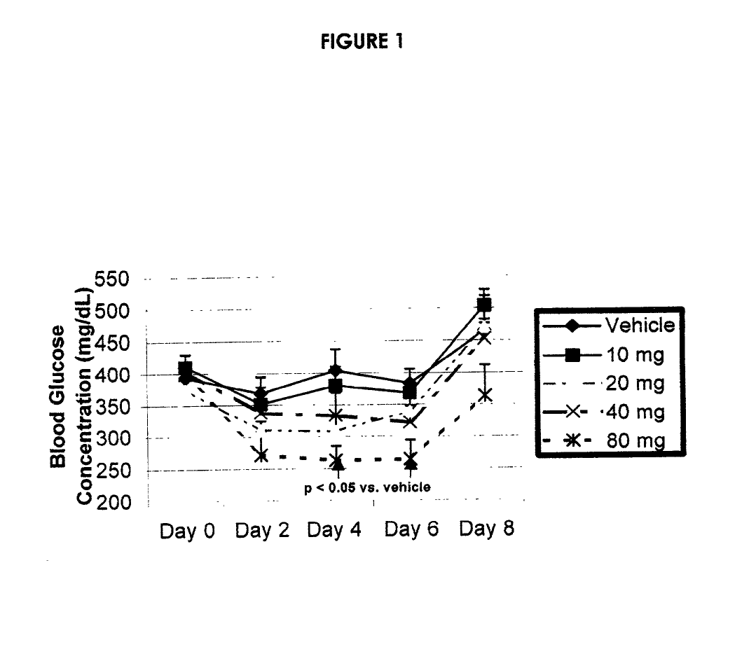

Diabetes was induced in Sprague-Dawley (SD) male rats (average body weight, 180 g) by IV injection of 60 mg streptozotocin. After 5 days, the average glucose concentration was in the range of about 350-400 mg/dL. Rats were then divided into five groups (n=7) and given either phosphate-buffered saline (PBS) (vehicle) or compound Ia (10, 20, 40 or 80 mg/kg of body weight) orally daily for 8 days. Treatment with Ia at a dose of 20, 40 or 80 mg/kg reduced the blood glucose concentrations of these rats (compared to those of vehicle-treated controls) from Day 2 through Day 6. The reduction was statistically significant (p<0.05) at Day 4 and Day 6 of the rats given 80 mg/kg. Results are shown in FIG. 1.

### EXAMPLE 4

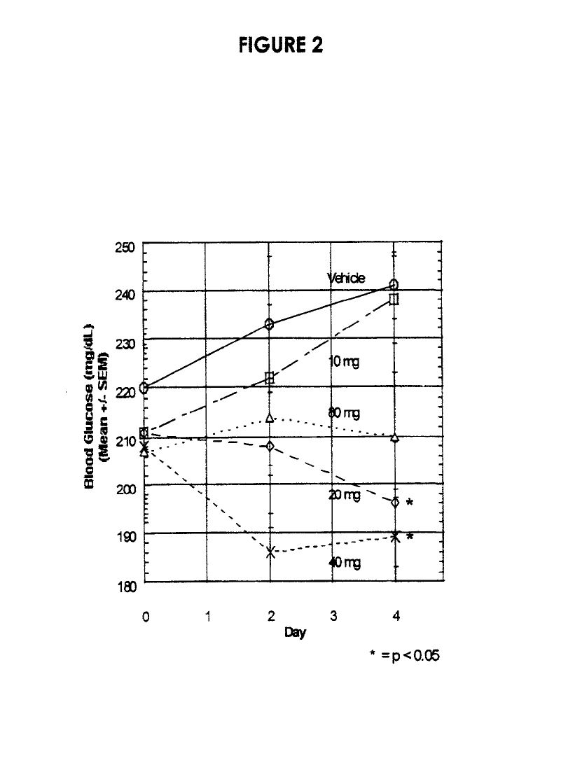

Obese (ob/ob) mice spontaneously develop diabetes, with glucose concentrations ranging between 200 and 300 mg/dL. In this experiment, Ia was administered daily in doses of 0 (vehicle only), 10, 20, 40, or 80 mg/kg of body weight to ob/ob mice for 4 days. By Day 4, the blood glucose concentrations of the animals given Ia were lower than those of the animals given the vehicle only; the differences were statistically significant for the groups given 20 and 40 mg/kg. Results shown in FIG. 2.

### EXAMPLE 5

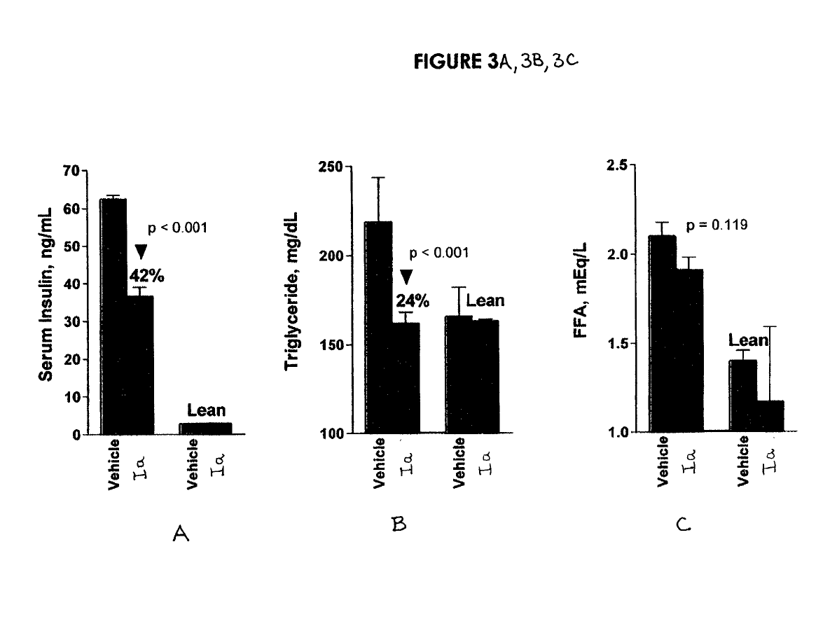

Serum concentrations of insulin, triglyceride, and free fatty acids (FFA) in ob/ob mice were measured on Day 8 following daily oral treatment with a dose of 20 mg/kg body weight for 7 days. Serum insulin concentrations were 42% lower in the Ia treated animals than they were in the vehicle-treated animals (A). Serum triglyceride concentrations were 24% lower in the Ia-treated mice than in the vehicle-treated mice (B). Serum FFA concentrations did not decrease significantly (C). Results shown in FIGS. 3A, B, C.

### EXAMPLE 6

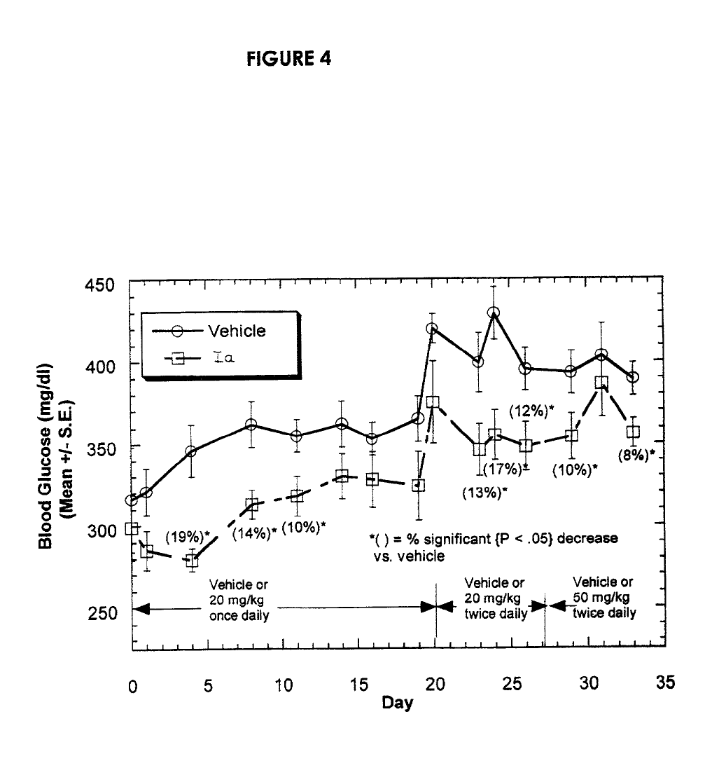

The ability of Ia to reduce glucose concentrations was examined further in db/db mice. Eight-week-old db/db mice with average blood glucose concentrations of 280-300 mg/dL were treated with vehicle or Ia (single 20-mg/kg doses daily, for 20 days; two 20-mg/kg doses daily for 8 days; and two 50-mg/kg doses daily for 5 days). Blood glucose concentrations in the mice given Ia were reduced 20% from those in the mice given the vehicle at the end of the first 20 days of treatment. Treatment with Ia at higher doses did not improve the glucose-lowering effect. Results shown in FIG. 4.

### EXAMPLE 7

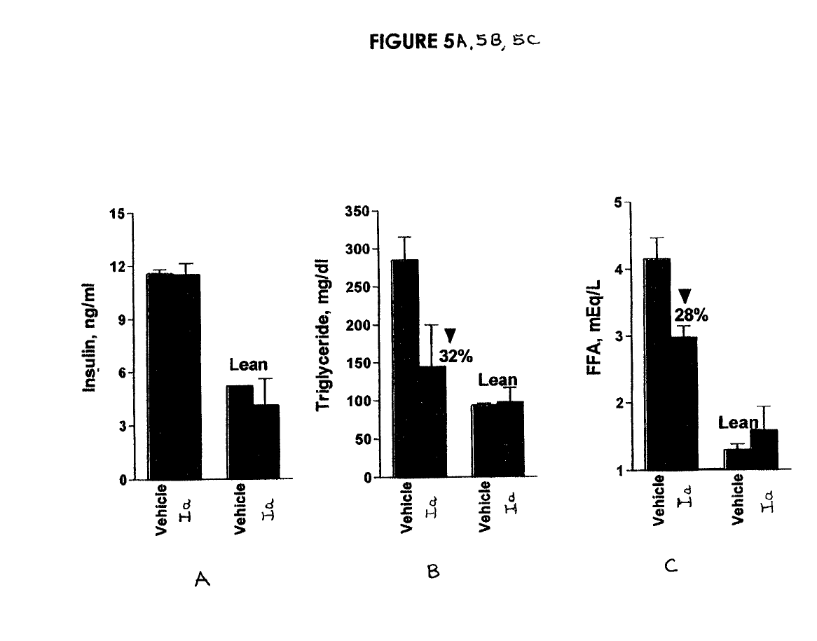

FIGS. 5A, B, C show the serum insulin, triglyceride, and free fatty acid (FFA) concentrations found in the db/db mice treated with Ia from the experiment shown in FIG. 4 (the analyses were done at the end of the experiment). Although the insulin concentrations in the IA-treated and vehicle-treated groups did not differ (A), the triglyceride (B) and FFA (C) concentrations were significantly lower in the mice treated with Ia than they were in the mice treated with vehicle, triglyceride concentrations were reduced 32% from those in the vehicle-treated mice, and free fatty acid concentrations were reduced 28% from those in the vehicle-treated mice.

### EXAMPLE 8

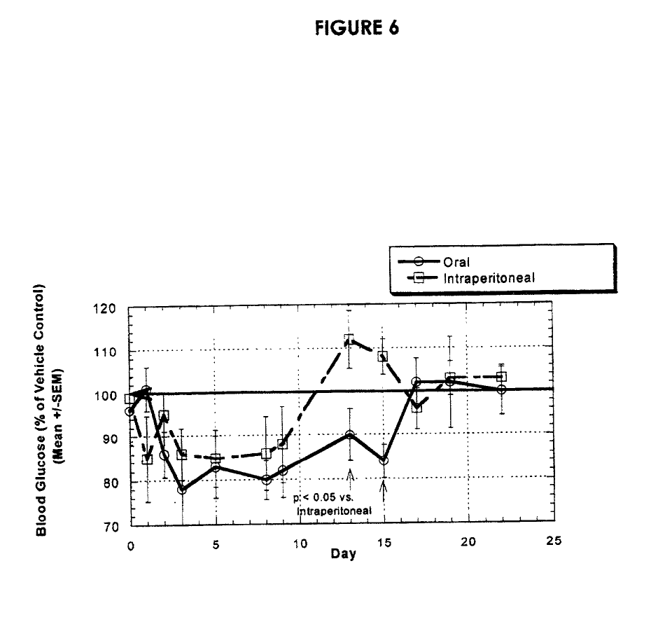

Compound Ia was administered either orally or intraperitoneally (IP) at a dose of 20 mg/kg to db/db mice for 22 days. After Day 9, the glucose-lowering effect of IP administered Ia disappeared, but that of orally administered Ia was maintained. The differences between the mice given oral Ia and those given IP Ia were statistically significant (p<0.05) on Days 13 and 15. Results shown in FIG. 6.

### EXAMPLE 9

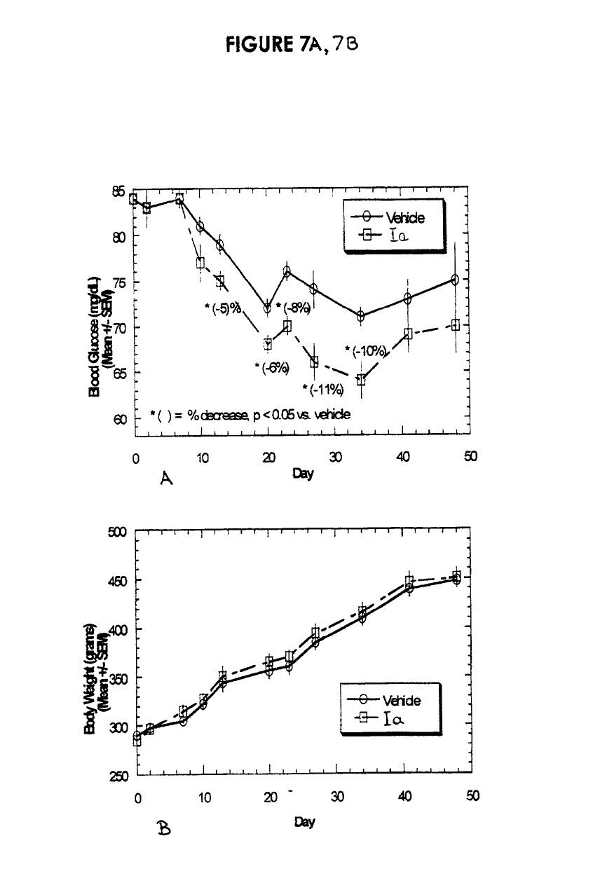

The effect of Ia was studied in female Zucker (fa/fa) rats (considered a good spontaneous genetic model of investigating insulin-resistant diabetes). Female fa/fa rats were given vehicle or Ia (20 mg/kg) daily for 58 days. (A) The blood glucose concentrations of the rats given Ia were lower than those of the rats given vehicle from Day 10 through the end of the experiment, and the differences were statistically significant on Days 9 through 34. (B) Throughout the experiment, the body weights of the rats in the two treatment groups were virtually identical. Results shown in FIGS. 7A, B.

### EXAMPLE 10

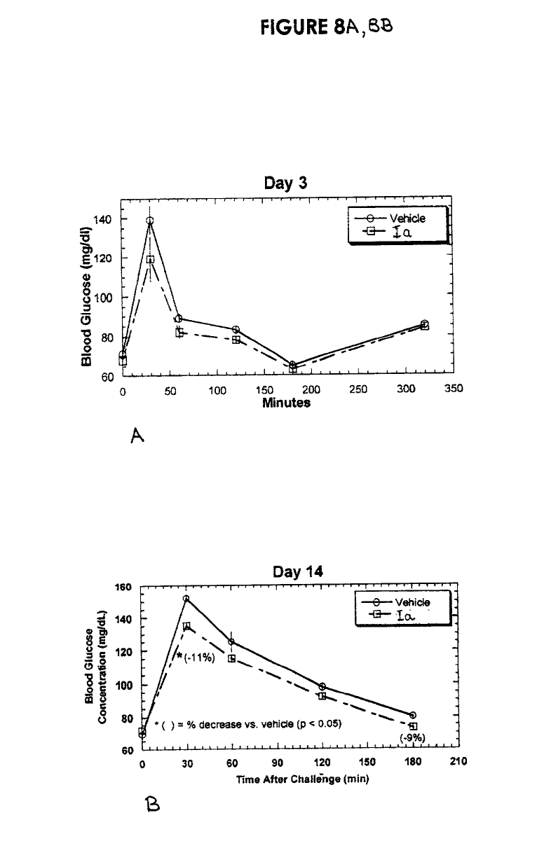

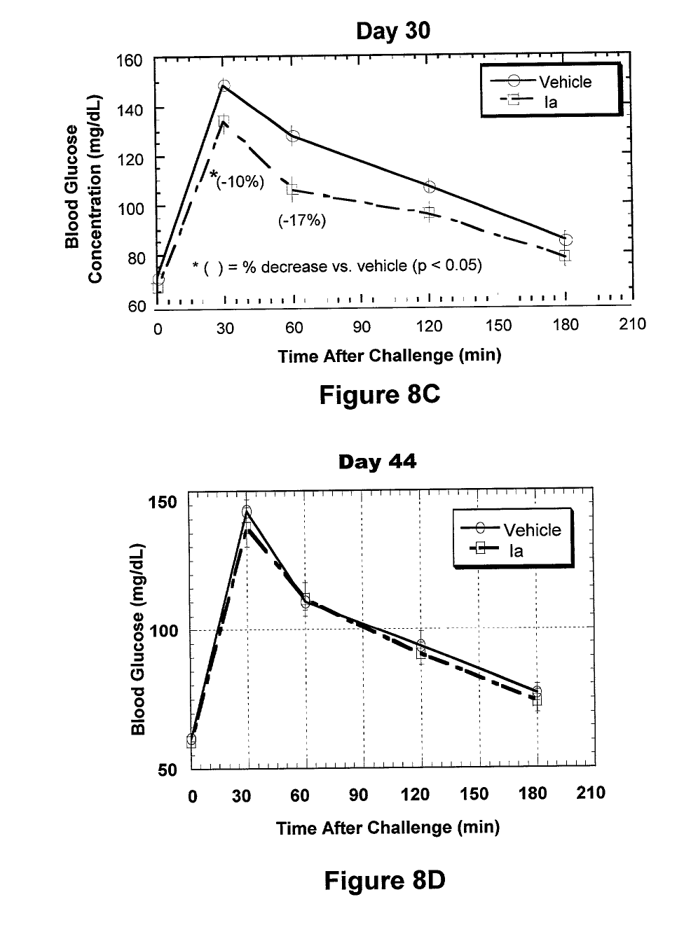

Zucker fa/fa rats were given vehicle or Ia (20 mg/kg) daily for 58 days; on Days 3, 14, 30, and 44, glucose tolerance tests (glucose, 2 mg/kg in water) were administered. The results of these tests show that the differences between treatment groups were statistically significant (p<0.05) 30 and 180 minutes after challenge on Day 14 (B) and 30 and 60 minutes after glucose challenge on Day 30 (C). By Day 44, the effect of Ia on glucose tolerance had disappeared. Results shown in FIGS. 8A-D.

### EXAMPLE 11

(A) The Zucker fa/fa rats treated with Ia (20 mg/kg) for 58 days (see FIG. 7) had serum insulin concentrations that were decreased 70-78% from those in the rats given vehicle only (p<0.05). This suggests that the mechanism by which Ia affects diabetes involves insulin-sensitization.

(B) In addition, the serum leptin concentrations of the IA-treated rats were 45% higher than those of the vehicle-treated rats. Results shown in FIGS. 9A, B.

### EXAMPLE 12

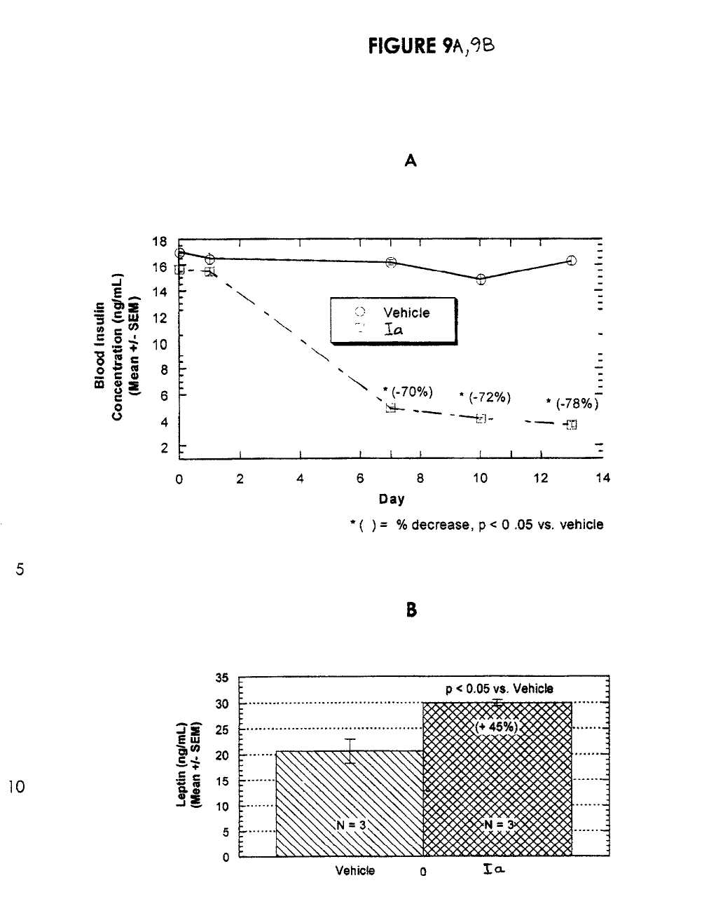

Serum concentrations of triglyceride, free fatty acid, and cholesterol were also measured in the Zucker fa/fa rats given vehicle or Ia (20 mg/kg) daily for 58 days (see also FIGS. 7 and 9). At the end of the study, the triglyceride, free fatty acid, and cholesterol concentrations found in the rats given Ia were reduced 70%, 89%, and 68% from those of the rats given vehicle. Results shown in FIG. 10.

### EXAMPLE 13

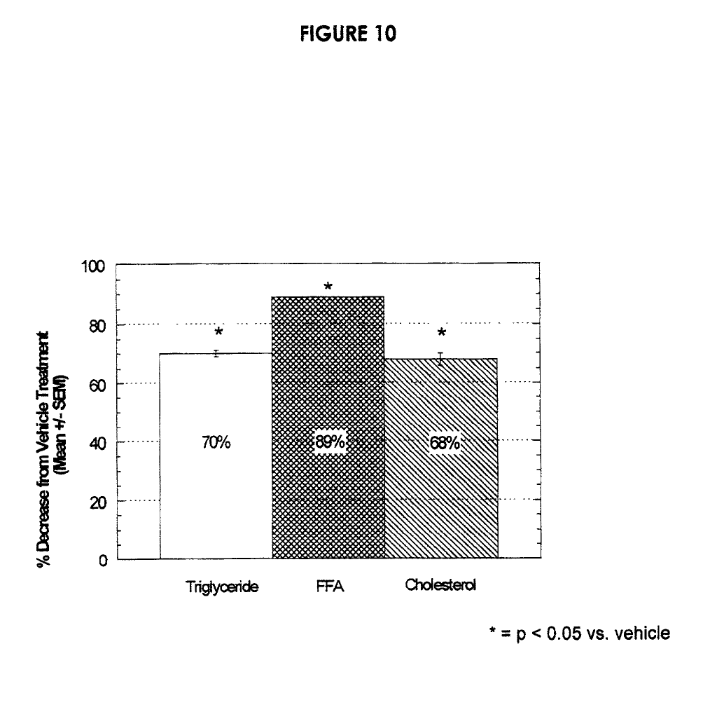

When the test described in connection with FIGS. 7-10 was conducted using male obese Zucker fa/fa rats treated with vehicle or Ia (20 mg/kg) daily for 65 days, the glucose concentrations, glucose tolerance, and leptin concentrations of the Ia-treated animals did not differ from those of the vehicle-treated animals. However, the insulin, triglyceride, free fatty acid, and cholesterol concentrations found in the Ia-treated rats at the end of the experiment were all lower than those found in the vehicle-treated animals. Results shown in FIGS. 11A-D.

### EXAMPLE 14

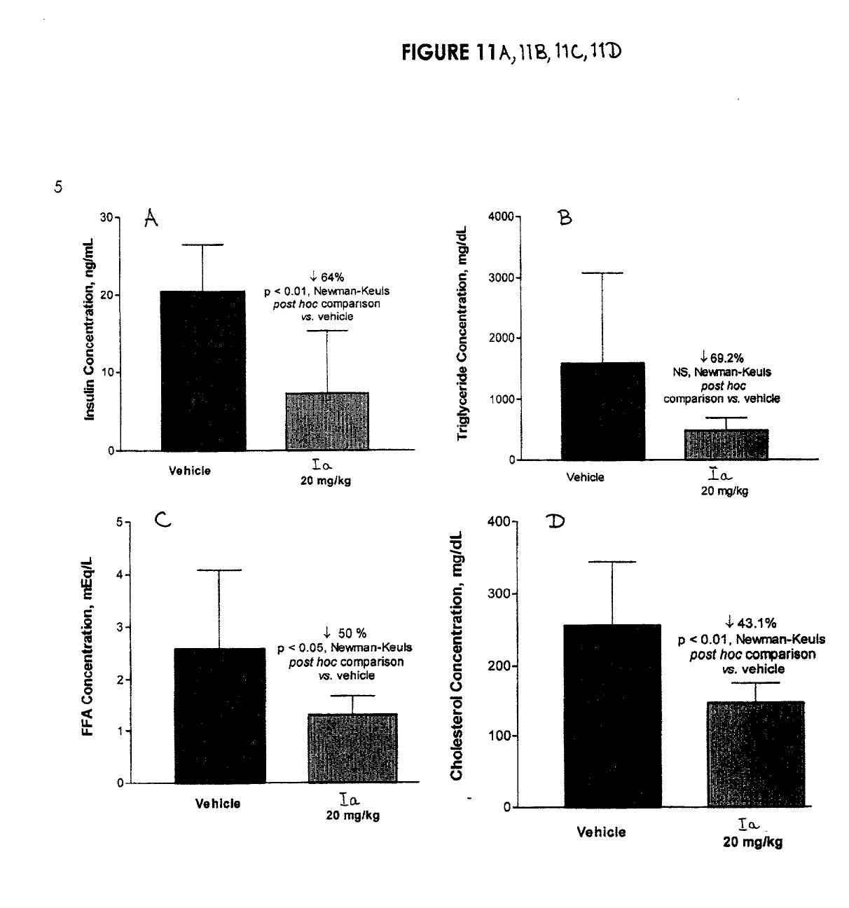

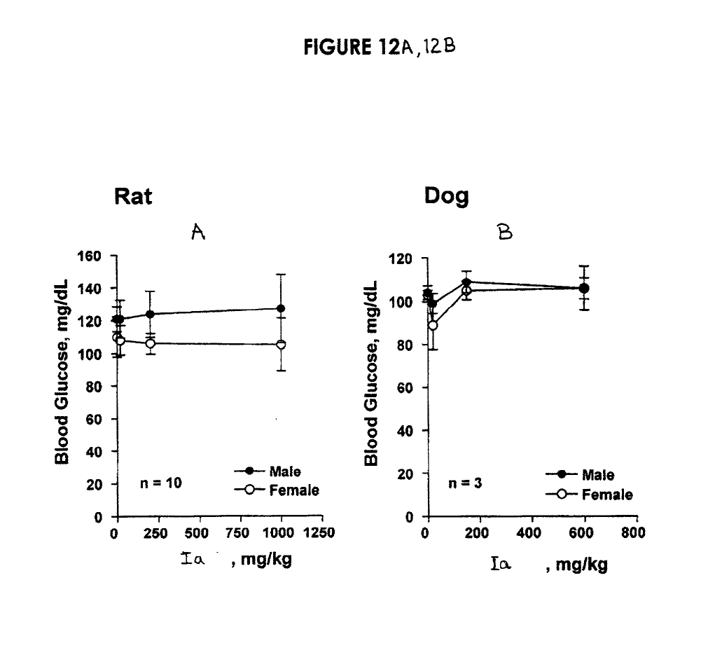

Compound Ia does not lower blood glucose concentrations in normal animals. This was demonstrated in two studies using rats and dogs. Daily oral administration of Ia for 28 days did not cause any hypoglycemic activity of this compound, even at very high doses (up to 1000 mg/kg). Results shown in FIGS. 12A, B.

### EXAMPLE 15

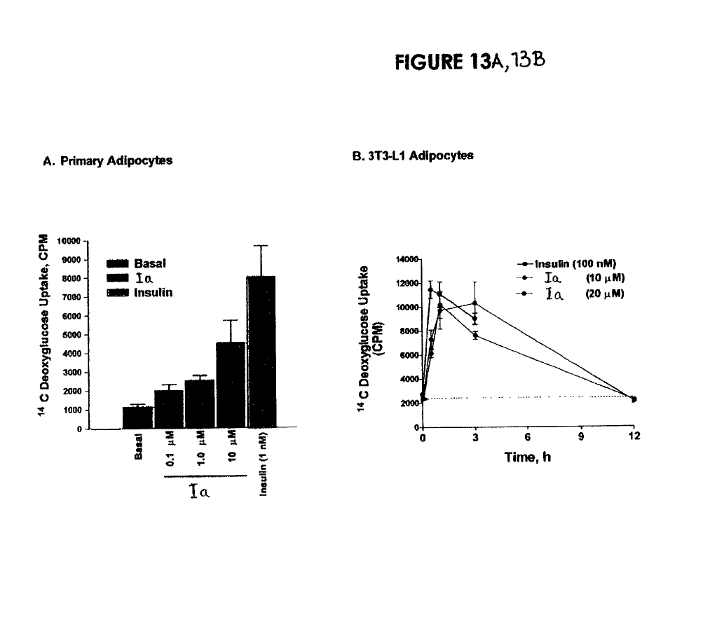

Glucose uptake was measured in normal adipocytes freshly prepared from the epididimal fat pad of Sprague-Dawley (SD) rats (170 g) in the presence of Ia at the indicated concentrations (A), and in differentiated 3T3-L1 adipocytes (B). Insulin was used as a positive control in both experiments. In both cases Ia stimulated glucose uptake in a manner similar to insulin. Results shown in FIGS. 13A, B.

### EXAMPLE 16

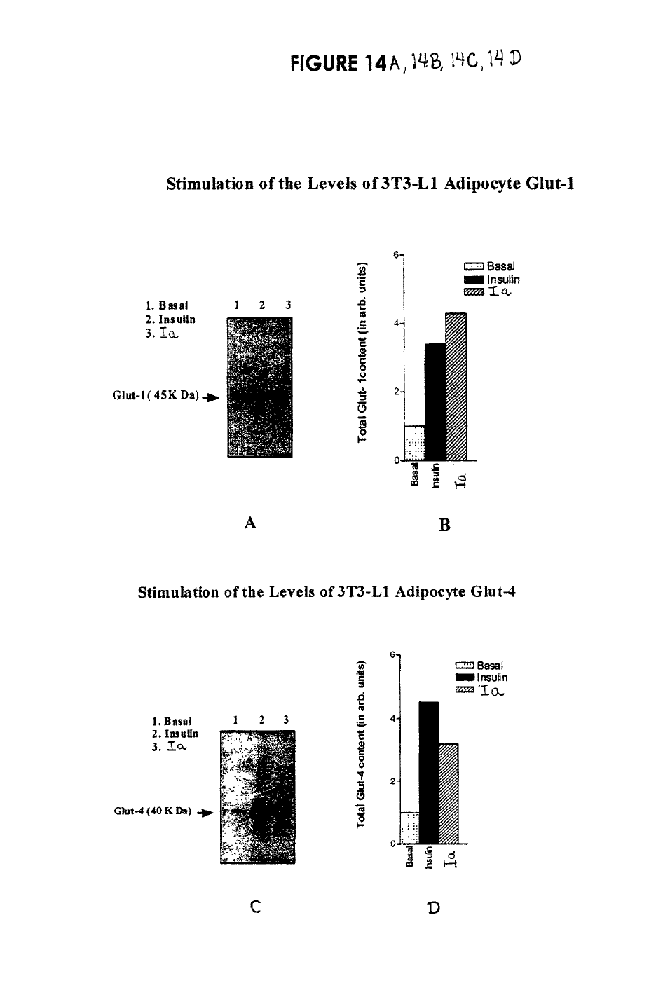

To determine whether Ia treatment affected expression of glucose transporters GLUT-1 and GLUT-4, differentiated 3T3-L1 adipocytes were treated with Ia, insulin or vehicle alone. Cells were lysed, subjected to 4-20% gradient SDS-PAGE, electroblotted, and probed with anti-GLUT-1 or anti-GLUT-4 monoclonal antibodies. As this figure shows, both GLUT-1 (A) and GLUT-4 (B) were up-regulated in 3T3-L1 cells following exposure of cells to Ia. Results shown in FIGS. 14A-D.

### EXAMPLE 17

Differentiated 3T3-L1 adipocytes were serum starved for 3 hours and then treated with either vehicle (medium alone) or Ia at a concentration of 10 μM for 30 minutes at 37° C. Cells were washed with phosphate-buffered saline (PBS), fixed with methanol at −20° C. for 20 minutes, rinsed with PBS three times, and incubated with PBS containing 10% calf serum for 30 min at 37° C. The slides were incubated with anti-GLUT-4 polyclonal antibody (1:50 dilution) in 10% calf serum for 2 hours at 37° C. Following this incubation, the slides were rinsed with PBS three times and then incubated with secondary antibody coupled with Alexa-Fluor (EXₘₐₓ 495 nm; Emₘₐₓ 519 nm) for 30 min. Finally, the slides were rinsed with PBS and mounted with prolonged antifade mounting media. The pictures generated using a Nikon confocal PCM 2000 microscope linked to an image analyzer showed high fluorescence in the Ia-treated cells. The fluorescence staining appeared in the cell membrane, indicating that treatment with Ia promoted translocation of GLUT-4 glucose transporters to the cell surface.

### EXAMPLE 18

Nine healthy male Swiss Webster mice were divided into three study groups of three. The first study group (FIG. 15A) received the compound of Ia at a dose of 16.7 mg/kg/BW, the second study group (FIG. 15B) received a dose of 167 mg/kg/BW, and the third study group (FIG. 15C) received a dose of 333 mg/kg/BW on day zero of the study. The mice were kept on regular food and water during the entire study period. During the study, the mice were under close observation and their behavior, gross physiology and mortality/survival were monitored. FIGS. 15A, 15B and 15C show that the survival rate in these mice in the course of the study period was 100%.

### EXAMPLE 19

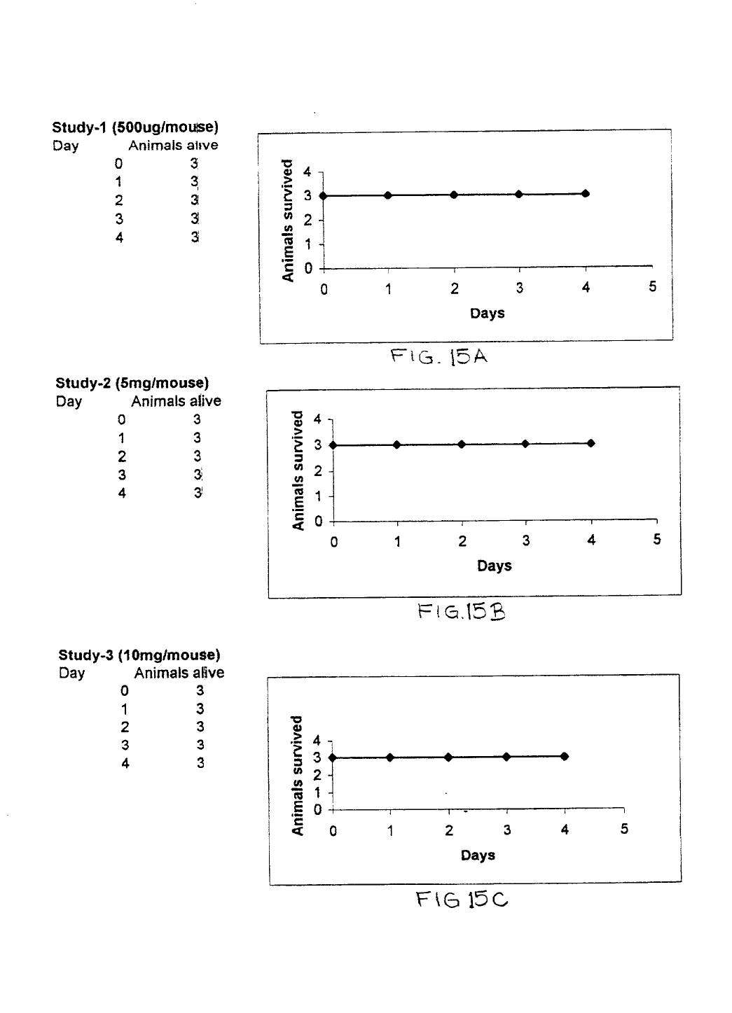

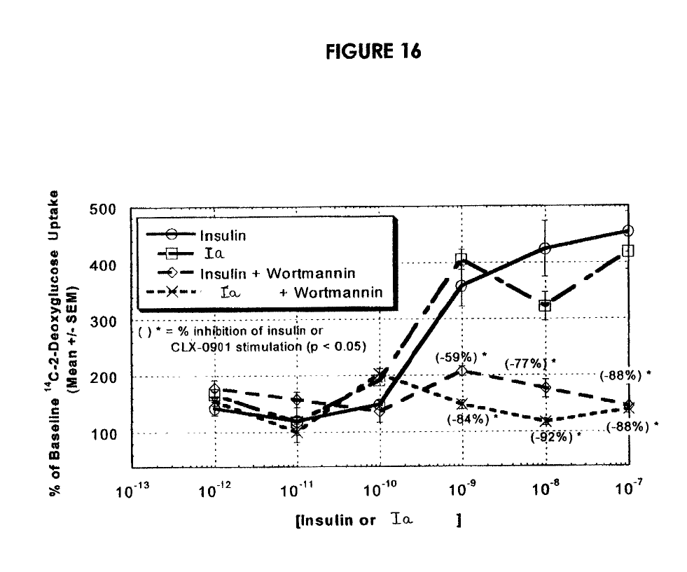

Wortmannin is a known inhibitor of phosphatidylinositol 3-kinase (PI 3-kinase), an enzyme required for the insulin-signaling pathway. In this experiment, the ability of wortmannin to inhibit Ia-stimulated glucose uptake was measured. Freshly prepared adipocytes were incubated with varying concentrations of either insulin or Ia, in the presence or absence of 4 μM wortmannin. The ability of adipocytes to take up glucose was then monitored using the ¹⁴C-deoxyglucose tracer. As shown here, treatment of adipocytes with wortmannin strongly inhibits insulin or Ia-dependent glucose uptake. This result suggests that Ia influences the PI 3-kinase pathway. Results shown in FIG. 16.

### EXAMPLE 20

Chinese hamster ovary cells that overexpress the human insulin receptor (CHO.IR cells) were grown in F12 Ham's medium with 10% fetal bovine serum (FBS) at 37° C. in 5% CO₂. Cells were serum-starved for 6 hours, and then incubated with vehicle, insulin (10 nM), or Ia (12.5, 25, or 50 μM) for 30 minutes at 37° C. Then the cells were washed with cold phosphate-buffered saline (PBS), and 13 μg of total cell lysates were separated by electrophoresis (4-20% SDS-PAGE), blotted onto a nitrocellulose membrane, and phosphorylation was detected with monoclonal antibody to phosphotyrosine (Transduction Laboratories, clone PY20). Western blots were developed using an enhanced chemiluminescence detection system, and the results were quantified by scanning then expressed as arbitrary units. The results indicate that Ia phosphorylates the insulin receptor and insulin-receptor substrate 1 in a dose-dependent manner (as does insulin). Results shown in FIG. 17.

### EXAMPLE 21

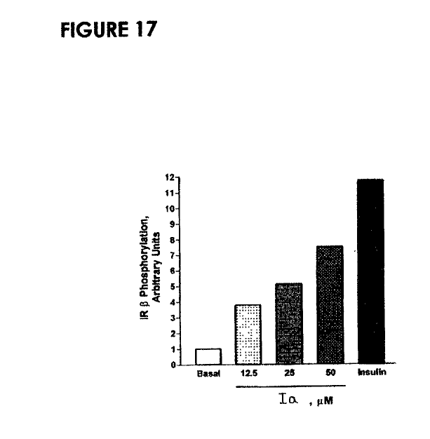

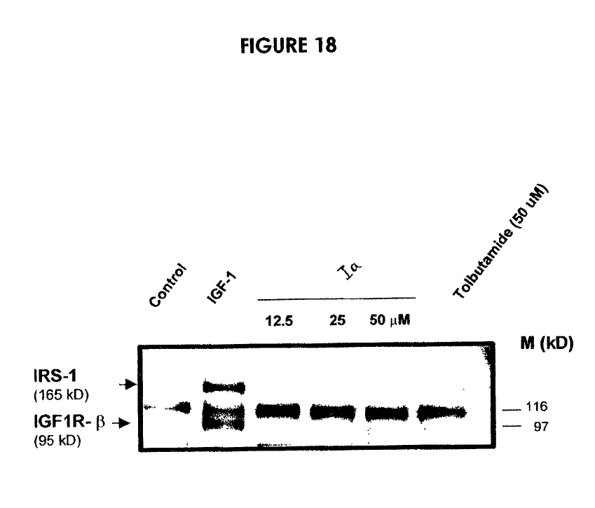

Chinese hamster ovary cells that overexpress the human insulin-like growth factor 1 receptor (CHO.IGF-1R cells) were grown in F12 Ham's medium with 10% FBS at 37° C. in 5% CO₂. Cells were serum-starved for 6 hours, and then incubated with vehicle, IGF-1 (100 nM), tolbutamide (50 μM), or Ia (12.5, 25, or 50 μM) for 30 minutes at 37° C. Then the cells were washed with cold phosphate-buffered saline (PBS), and 21 μg of total cell lysates were separated by electrophoresis (4-20% SDS-PAGE) and blotted onto a nitrocellulose membrane; phosphorylation was detected with monoclonal antibody to phosphotyrosine (Transduction Laboratories, clone PY20). The results show that Ia does not phosphorylate the insulin-like growth factor 1 receptor or insulin receptor substrate 1 in CHO.IGF-1R cells. Results shown in FIG. 18.

### EXAMPLE 22

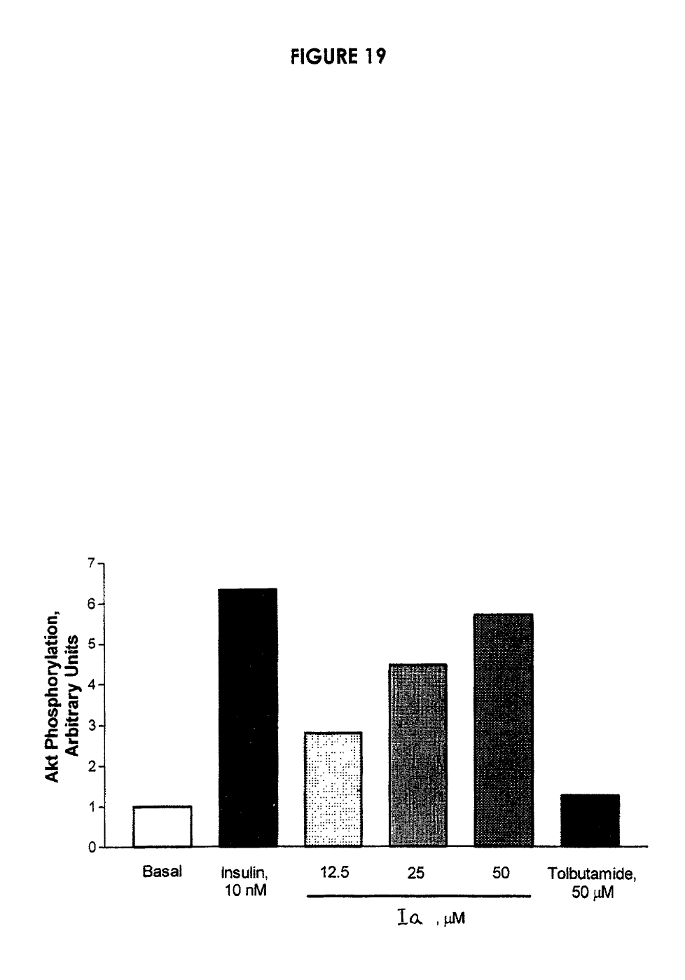

Chinese hamster ovary cells that overexpress the human insulin receptor (CHO.IR) were grown in F12 Ham's medium with 10% fetal bovine serum (FBS) at 37° C. in 5% CO₂. Cells were serum starved for 6 hours and incubated with vehicle, insulin (10 nM), tolbutamide (50 μM), or one of 3 different doses of Ia (12.5, 25, or 50 μM) for 30 min at 37° C. Then the cells were washed with cold phosphate-buffered saline (PBS), and 25 μg of total cell lysates were separated by electrophoresis (4-20% SDS-PAGE), blotted onto a membrane, and detected with the antibody (A) anti Phospho-Akt (Ser 473) (New England Biolabs). Western blots were developed using an enhanced chemiluminescence detection system, and the results were quantified by scanning and then expressed as arbitrary units. The results indicate that there was a dose-dependent increase in phosphorylation of Akt in the presence of Ia. Results shown in FIG. 19.

### EXAMPLE 23

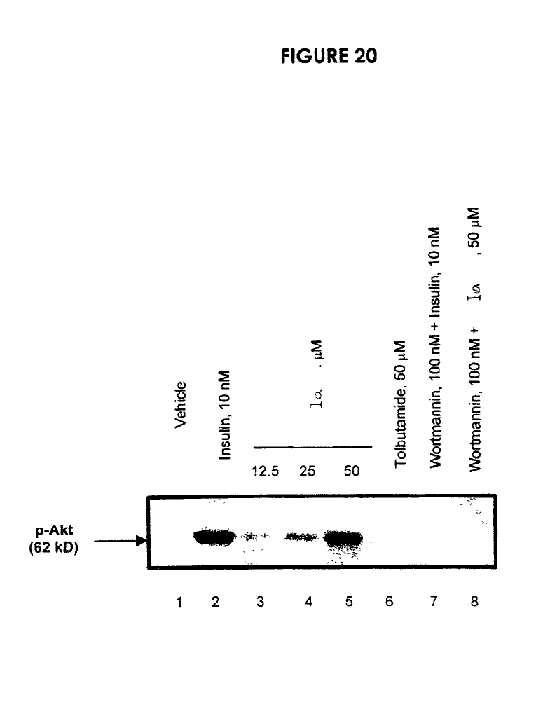

Chinese hamster ovary (CHO) cells that had been serum-starved for 6 h were incubated with vehicle, insulin (10 nM), tolbutamide (50 μM), or one of three doses of Ia (12.5, 25, or 50 μM) for 30 minutes at 37° C. Two groups had been preincubated with 100 nM Wortmannin (Lanes 7 and 8), and had the insulin (10 nM) and Ia (50 μM) added at this time. Then cells were washed with cold phosphate-buffered saline (PBS), and 20 μg of total cell lysates were separated by electrophoresis (4-20% SDS-PAGE) and blotted onto a membrane; Akt-phosphorylation was detected with an antibody [anti Phospho-Akt (Ser 473), New England Biolab]. The results show that Wortmannin inhibited the Akt-phosphorylation stimulated by insulin and by Ia. Results shown in FIG. 20.

### EXAMPLE 24

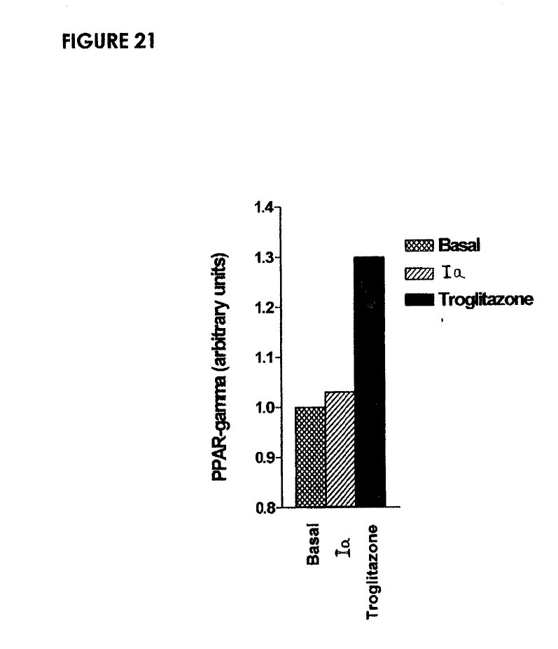

All thiazolidinedione compounds are known to stimulate glucose uptake via a mechanism that involves binding to and increasing the expression of a nuclear receptor transcription factor known as peroxisome proliferator activated receptors (PPAR-γ). To determine whether Ia up-regulates glucose uptake by a mechanism that involves PPAR-γ, 3T3-L1 adipocytes were incubated with vehicle, Ia (5 μM), or troglitazone (5 μM), for 48 hours. PPAR-γ expression was assessed by immunoblotting. Western blots were developed using an enhanced chemiluminescence detection system, and the results were quantified by scanning and then expressed as arbitrary units. The results in the figure show that troglitazone induced an increase in PPAR-γ, while Ia did not induce an increase in PPAR-γ over the basal level of this transcription factor. Results shown in FIG. 21.

### EXAMPLE 25

Differentiation of fibroblasts to adipocytes involves the expression of PPAR-γ. All members of the thiazolidinedione class of antidiabetic compounds stimulate PPAR-γ expression and promote the differentiation of fibroblasts to adipocytes. Similarly, insulin also stimulates the differentiation of fibroblasts to adipocytes. To examine the effect of Ia on this differentiation process, 3T3-L1 fibroblasts were incubated with Ia (1 μM), insulin (0.17 mM) or a combination of both. Following incubation, the cells were lysed, and the quantity of expressed PPAR-γ was analyzed by ECL blot analysis using anti-PPAR-γ antibody. Treatment of fibroblasts with Ia did not enhance the differentiation process. In a positive control, insulin treatment of fibroblasts stimulated the differentiation of these cells to adipocytes in association with increased levels of PPAR-γ.

### EXAMPLE 26

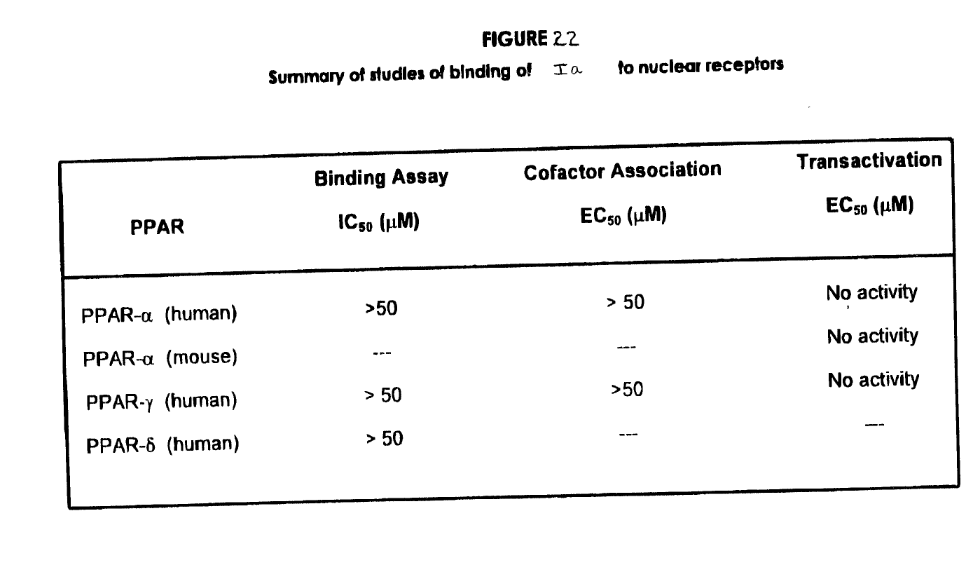

FIG. 22 shows the results of three tests conducted to determine whether or not Ia is an agonist of nuclear PPAR receptors. The ability of Ia to bind human recombinant PPAR-α, PPAR-γ, or PPAR-δ was shown using a radioligand-binding assay that measures the displacement of an established radiolabeled ligand. In this assay, the IC, values for all three nuclear receptors were greater than 50 μM. Ligand induced conformational changes in PPAR are known to promote the binding of coactivator molecules. The cofactor association was measured by the time-resolved fluorescence (HTRF) assay that uses energy transfer between two adjacent molecules to measure the ability of Ia to promote the association of PPARs with cofactor proteins. Ia did not induce any association of cofactors. Finally, a cell-based transactivation functional assay was performed to determine the effect of Ia on PPARs in a biological system. In this experiment, COS cells with chimeric receptors were treated with Ia, and the transcription activity was measured by an increase in luciferase activity. No activation of PPARs by Ia was observed. All of these results confirm that Ia is not an agonist of these PPARs.

### EXAMPLE 27

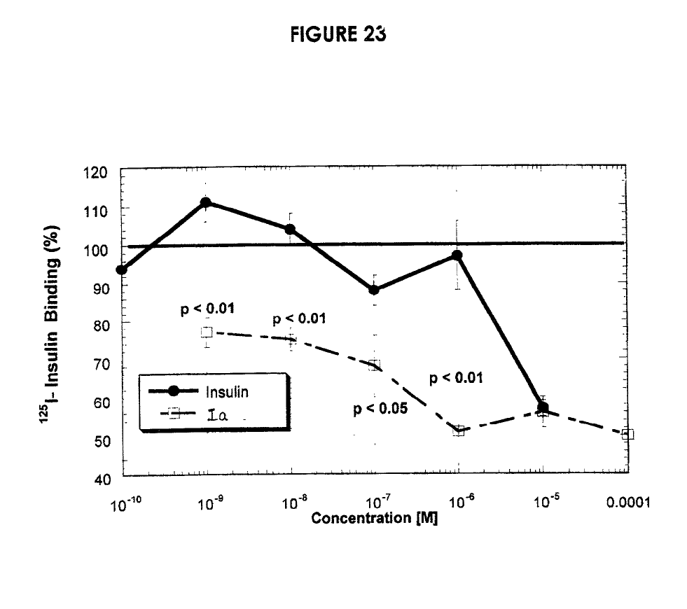

Differentiated 3T3-L1 cells (in triplicate wells) were treated with either Ia or cold insulin for one hour at 37° C. at the indicated concentrations. After incubation, excess compounds were washed away, and the cells were incubated with a fixed amount of ¹²⁵I-insulin (10 pM; 2000 Ci/mmol) for 12 hours at 4° C. The cells were washed and then lysed with 0.1% SDS and counted in a scintillation counter. As expected, increasing the dose of cold insulin inhibited the binding of radioactive insulin, while a 45% inhibition occurred with pre-incubation with Ia. Results shown in FIG. 23.

### EXAMPLE 28

Real-time direct binding of Ia to the insulin receptor was demonstrated by using a Biocore 3000 (which measures surface plasmon resonance). The intensity and wavelength of light reflected off a metal surface with a thin film of solution on it is affected by the mass concentration of components at the liquid-surface interface. The interaction of molecules in the liquid phase alters the intensity of the reflected light at a particular angle. In this experiment, purified insulin receptors containing both alpha and beta subunits were immobilized into the sensor surface of flow cell 2 of a Biocore 3000 with a gold film; flow cell 1 was used as a control for background. When Ia was injected at concentrations of 200 μM, 100 μM and 10 μM, a response indicative of binding to insulin receptor (binding curve) was seen within a few seconds, which is similar to the binding curves obtained with insulin.

### EXAMPLE 29

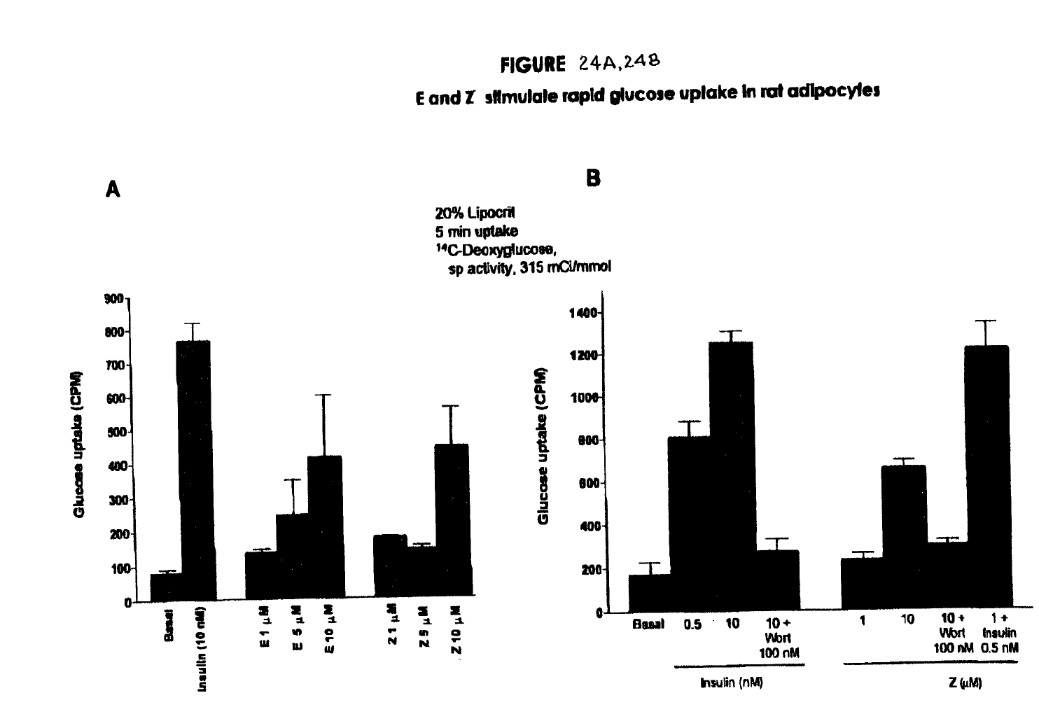

Glucose uptake was measured in normal adipocytes freshly prepared from the epididimal fat pad of SD rats in the presence of E or Z isomers of Ia or Ib. After the cells were preincubated with the isomers at the indicated concentrations for 30 min. ¹⁴C-deoxy glucose was added, and the preparations were incubated for an additional 5 min. FIG. 24A shows the extent of glucose uptake stimulated by the two isomers was similar. FIG. 24B shows the stimulatory effect of the Z form (Ib) was additive to that of insulin, and the effect was blocked by Wortmannin (15 minute preincubation), as was shown earlier for the E form of (Ia) (see FIG. 16).

### EXAMPLE 30

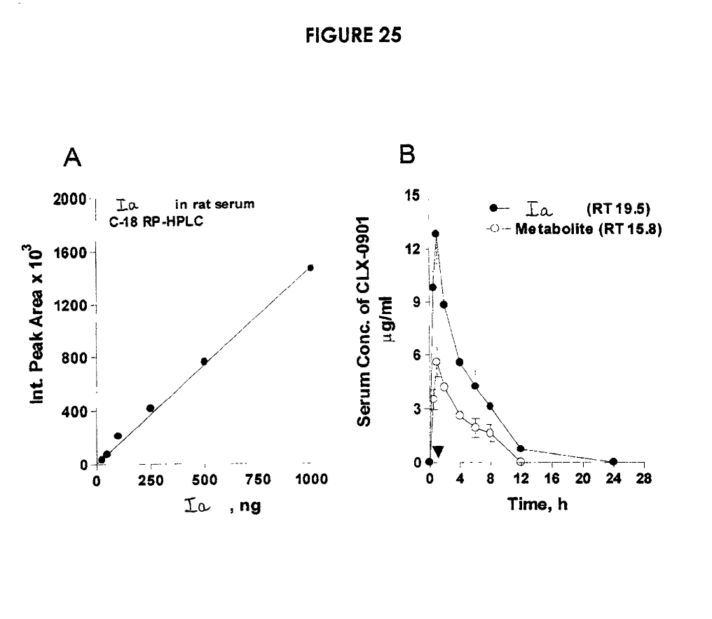

A highly sensitive method for detecting Ia in Sprague-Dawley (SD) rat serum was developed in which Ia can be detected at a level of 10-25 ng. The kinetics of drug absorption and clearance from the circulation were studied in a rat model. SD rats were given oral doses of Ia (20 mg/kg). At different time intervals, blood was collected and serum was analyzed for IA. As shown in the figure, Ia was absorbed maximally at 1 hour following oral delivery of the drug and is cleared from the circulation by 24 hours. Results shown in FIGS. 25A, B.

### EXAMPLE 31

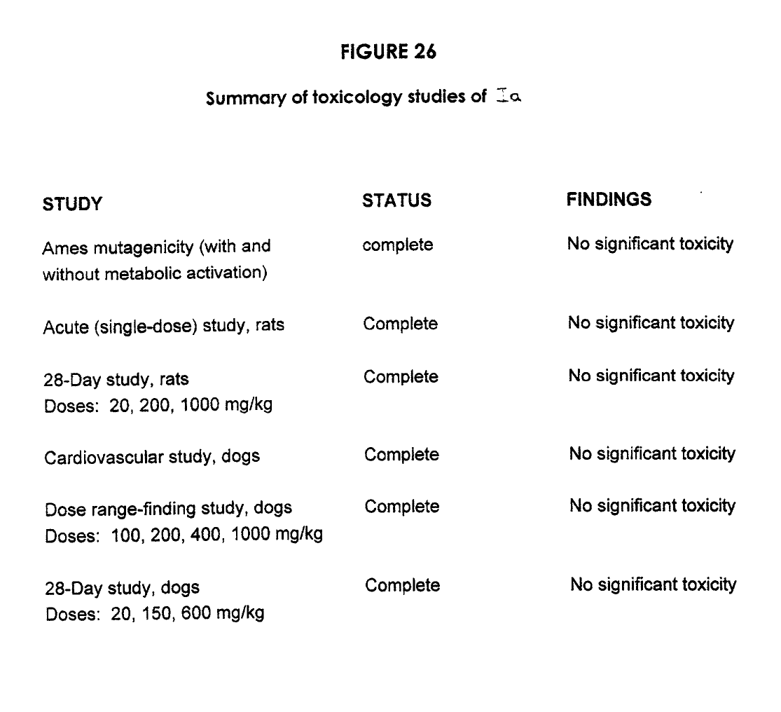

Various toxicology studies have been conducted, and their status and results are summarized in FIG. 26. Doses as high as 1000 mg/kg have been administered, and no serious toxicity issues have been uncovered.

What is claimed:

1. A compound of the formula I: 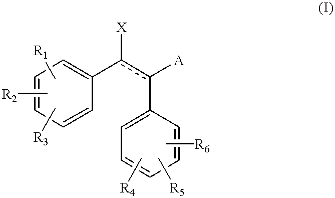
wherein the bond represented by the dotted line may be an optional double bond, and the geometry across the bond may be E or Z;
A=—COOR, —CONR′R″, —CN, —COR₇ wherein R, R′, R″ and R₇ are defined below;
X=H, OH, or C₁-C₁₀ linear or branched alkyl or alkenyl groups, optionally substituted with COOR, carbonyl, or halo;
R=H or C₁-C₂₀ linear or branched alkyl or aryl or aralkyl, or a pharmaceutically acceptable counter-ion;
R₁, R₂, R₃, R₄, R₅, R₆ and R₇ are independently H; C₁-C₂₀ linear or branched alkyl or alkenyl groups optionally substituted; COOR where R is as defined previously; NR′R″ or CONR′R″, where R′ and R″ may be independently H or C₁-C₂₀ linear or branched alkyl or aryl; OH; C₁-C₂₀ alkoxy; C₁-C₂₀ acylamino; C₁-C₂₀ acyloxy; C₁-C₂₀ alkanoyl; C₁-C₂₀ alkoxycarbonyl; halo; NO₂; SO₂R′″; CZ₃, where each Z is independently a halo atom, H, alkyl, chloro or fluoro-substituted alkyl; or SR′″, where R′″ may be H or linear or branched C₁-C₂₀ alkyl; or R₂ and R₃ together, or R₅ and R₆ together may be joined to form methylenedioxy or ethylenedioxy groups;
with the proviso that when X, R₃, R₅ and R₆ are H; R₄ is p-hydroxy; R₁ and R₂ together are 3,5-dimethoxy; then the dotted line is not a double bond in the E-configuration.

2. A compound according to claim 1 wherein A=—COOR.

3. A compound of the formula II: 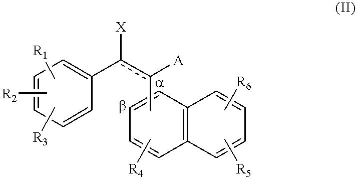
wherein the bond represented by the dotted line may be an optional double bond, the geometry across the bond may be E or Z, and the naphthyl group may be linked at an α or β position;
A=—COOR; —CONR′R″, —CN, —COR₇ wherein R, R′, R″ and R₇ are defined below;
X=H, OH, or C₁-C₁₀ linear or branched alkyl or alkenyl groups, optionally substituted with COOR, carbonyl, or halo;
R=H or C₁-C₂₀ linear or branched alkyl or aryl or aralkyl, or a pharmaceutically acceptable counter-ion;
R₁, R₂, R₃, R₄, R₅, R₆, and R₇ are independently H; C₁-C₂₀ linear or branched alkyl or alkenyl groups optionally substituted; COOR where R is defined previously; R; NR′R″ or CONR′R″, where R′ and R″ may be independently H or C₁-C₂₀ linear or branched alkyl or aryl; OH; C₁-C₂₀ alkoxy; C₁-C₂₀ acylamino; C₁-C₂₀ acyloxy; C₁-C₂₀ alkanoyl; C₁-C₂₀ alkoxycarbonyl; halo; NO₂; SO₂R′″; CZ₃; where each Z is independently a halo atom, H, alkyl, chloro or fluoro-substituted alkyl; or SR′″, where R′″ may be H or linear or branched C₁-C₂₀ alkyl or R₂ and R₃ together, or R₅ and R₆ together may be joined to form metheylenedioxy or ethylenedioxy groups.

4. A compound according to claim 1, wherein A=—COOR, X, R₃, R₅ and R₆ are H; R₄ is p-hydroxy; R₁ R₂ together are 3,5-dimethoxy; and the dotted line is a double bond in the Z-configuration.

5. A compound according to claim 4, wherein R is H.

6. A compound according to claim 4, wherein R is Na+.

7. A compound according to claim 2, wherein R₄ is p-hydroxy; R₁ and R₂ together are 3,5-dimethoxy and the dotted line represents a double bond.

8. A compound according to claim 3, wherein R₁ and R₂ together are 3,5-dimethoxy and the dotted line represents a double bond.

9. A pharmaceutical composition for the treatment of diabetes comprising a therapeutically effective amount of a compound of any one of the claims 1 to 8, or mixtures thereof, in a pharmaceutically acceptable carrier.

10. A composition according to claim 9 which is suitable for oral administration.

11. A method for treating diabetes comprising the step of administering to a subject suffering from a diabetic condition a therapeutically effective amount of a compound according to any one of claims 1 to 8, or mixtures thereof, in a pharmaceutically acceptable carrier.

12. A method according to claim 11 in which said compound is administered orally to said subject.

13. A pharmaceutical composition for the treatment of diabetes comprising a therapeutically effective amount of a compound according to any of claims 1 to 8 in a physiologically acceptable carrier, wherein the bond represented by the dotted line may be an optional double bond, and the geometry across the bond may be E or Z;
R=H, linear or branched C₁-C₂₀ alkyl, aryl or aralkyl, or a pharmaceutically acceptable counter-ion.

14. A composition according to claim 13, wherein R is H or Na+ and said double bond is in the E-configuration.

15. A composition according to claim 13, wherein R is H or Na+ and said double bond is in the Z-configuration.

16. A composition according to claim 15, wherein R is Na+.

17. A composition according to claim 14, wherein R is Na+.

18. A composition according to claim 13, wherein said composition is suitable for oral administration.

19. A method of treating diabetes comprising a step of administering to a subject suffering from a diabetic condition a therapeutically effective amount of a compound according to any of claims 1 to 8 in a physiologically acceptable carrier, wherein the bond represented by the dotted line may be an optional double bond, and the geometry across the bond may be E or Z;
R=H, linear or branched C₁-C₂₀ alkyl or aryl, or a pharmaceutically acceptable counter-ion.

20. A method according to claim 19, wherein R is H or Na+ and said double bond is in the E-configuration.

21. A method according to claim 19, wherein R is H or Na+ and said double bond is in the Z-configuration.

22. A method according to claim 20, wherein R is Na+.

23. A method according to claim 21, wherein R is Na+.
